(19) United States
(12) Patent Application Publication
(10) Pub. No.: US 2004/0193095 A1
(43) Pub. Date: Sep. 30, 2004

(54) Implants for treating ocular hypertension, methods of use and methods of fabrication

(76) Inventor: John H. Shadduck, Tiburon, CA (US)

Correspondence Address: John H. Shadduck, 1490 Vistazo West, Tiburon, CA, 94920, US

(21) Appl. No.: 10/759,797

(22) Filed: Jan. 17, 2004

Related U.S. Application Data
(60) Provisional application No. 60/459,196, filed on Mar. 29, 2003.

(51) Int. Cl. A61F 2/14

(52) U.S. Cl. 604/8; 623/4.1; 623/23.7

(57) ABSTRACT

An implantable stent and method for treating ocular hypertension. In a preferred embodiment, the stent is of a shape memory polymer that has at least one expandable inflow or outflow end. The stent is introduced in a minimally invasive procedure with an inflow end positioned generally within the uveoscleral plane. In various embodiments, the outflow end of the stent is configured to extend within the subconjunctival plane generally inward of the lymphatic vessel network. A method of the invention for controlling intraocular pressure (IOP), includes directing outflows into the lymphatic vessel network, wherein the lymphatic system then will naturally controls outflows, IOP and prevent excessive lowering of intraocular pressure.

### CROSS-REFERENCE TO RELATED APPLICATIONS

This application is claims benefit of Provisional U.S. Application Ser. No. 60/459,196 filed Mar. 29, 2003 (Docket No. S-AEG-004) titled Implantable Stent and Methods for Treating Glaucoma, which is incorporated herein by this reference.

### BACKGROUND OF THE INVENTION

1. Field of the Invention

A stent or implant body and method for reducing ocular hypertension, and more in particular, a stent device for aqueous outflow bypass to the eye's subconjunctival lymphatic system from the interior of the eye in the region of the uveoscleral plane, ciliary body and anterior chamber.

2. Description of the Related Art

Glaucoma is the second leading cause of legal blindness in the United States with approximately 80,000 people in the United States being legally blind as a result of glaucoma. Large numbers of people suffer lesser visual impairment, with the American Academy of Ophthalmology reporting that approximately 2 million persons in the United States have primary open angle glaucoma (POAG, a common form of glaucoma). As many as seven million office visits annually in the U.S. for glaucoma diagnosis and treatments.

Glaucomas are a group of eye diseases that are characterized by elevated intraocular pressure (IOP) that causes a pathological change in nerve fiber layers of the retina resulting in losses in the field of vision. In a healthy eye, the ciliary body produces aqueous humor which circulates from the posterior chamber to the anterior chamber. The aqueous flows outwardly and exits the anterior chamber through the trabecular meshwork and the Schlemm's canal, which is located about the periphery of the anterior chamber, as well as through the region of the uveoscleral plane. If the aqueous outflow paths, in particular relating to Schlemm's canal are, not functioning properly, an excess of aqueous humor will be present in the anterior chamber the intraocular pressure (IOP) may rise. The increased IOP associated with decreased aqueous outflows result in glaucoma and can thus lead to blindness.

Normal intraocular pressure is considered to be less than about 21-22 mm. Hg. However, as many as one in six patients with glaucoma have pressure below 21-22 mm. Hg and yet still have progressive eye damage. Further, in any single diagnostic test, as many as one half of glaucoma patients will exhibit normal IOP levels but will actually will average to have IOPs that are greater than 21-22 mm. Hg. Various surgical procedures and implant devices have been developed for treating glaucoma by increasing the rate of outflows of aqueous humor from the anterior chamber. None of the outflow devices have been widely accepted, and most require invasive surgery.

What is needed is a reliable implant device and method for treating high intraocular pressure. In particular, what is needed is an implantable device that can be implanted in a simplified, minimally invasive procedure. Of particular importance, it would be desirable to have an implant that is inexpensive and that can be implanted by health care personnel world-wide that does not require highly specialized surgical skills. A large number of glaucoma patients world-wide do not have access to IOP-lowering drugs or expensive glaucoma surgeries.

### BRIEF DESCRIPTION OF THE DRAWINGS

FIG. 1 is a perspective cut-away view of an eye with an exemplary stent or implant body extending between the eye's lymphatic network in the subconjunctival plane to a region of the uveoscleral plane.

FIG. 2A is a perspective view the stent of FIG. 1 fabricated at least in-part of a shape memory polymer (SMP) in a temporary reduced cross-sectional shape.

FIG. 2B is a perspective view of the SMP stent of FIG. 2A in its memory shape.

FIG. 3 is a sectional view of a microfabricated structure of a polymer stent as in FIG. 2B that is microfabricated by soft lithography means.

FIG. 4A is schematic view of a shape memory polymer end portion of an alternative stent with a biodegradable SMP component for expanding a branched outflow or inflow network.

FIG. 4B is view of the end portion of the stent of FIG. 4A after the biodegradable SMP has been absorbed by the body.

FIG. 5 is perspective sectional view of an eye with another embodiment of a stent extending between the subconjunctival plane proximate the eye's lymphatic network and the anterior chamber.

FIGS. 6A-6C are schematic views of a method of implanting a flexible polymer stent in the eye with an outflow end in the subconjunctival plane proximate the eye's lymphatic network.

FIG. 7 is perspective view of an alternative stent that is configured to extend substantially perpendicularly from the region of the eye's lymphatic network in the subconjunctival plane to a region of the uveoscleral plane.

FIG. 8A is perspective sectional view of an eye illustrating the stent of FIG. 7 as it is implanted with the SMP (distal) end in a temporary compacted shape.

FIG. 8B is view of the eye as in FIG. 8A with the SMP (distal) end of the stent in a memory expanded shape.

### DESCRIPTION OF PREFERRED EMBODIMENTS OF THE INVENTION

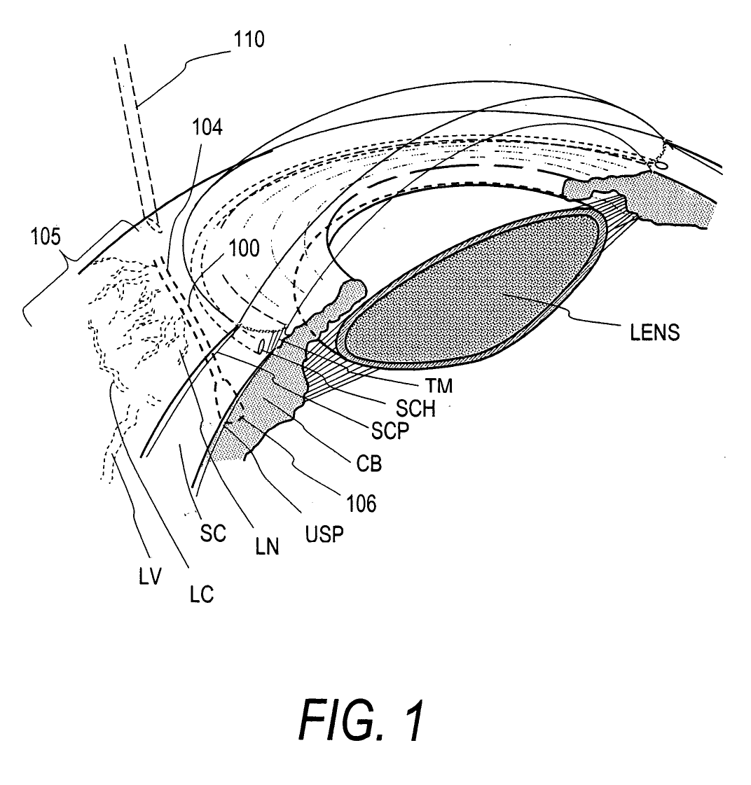

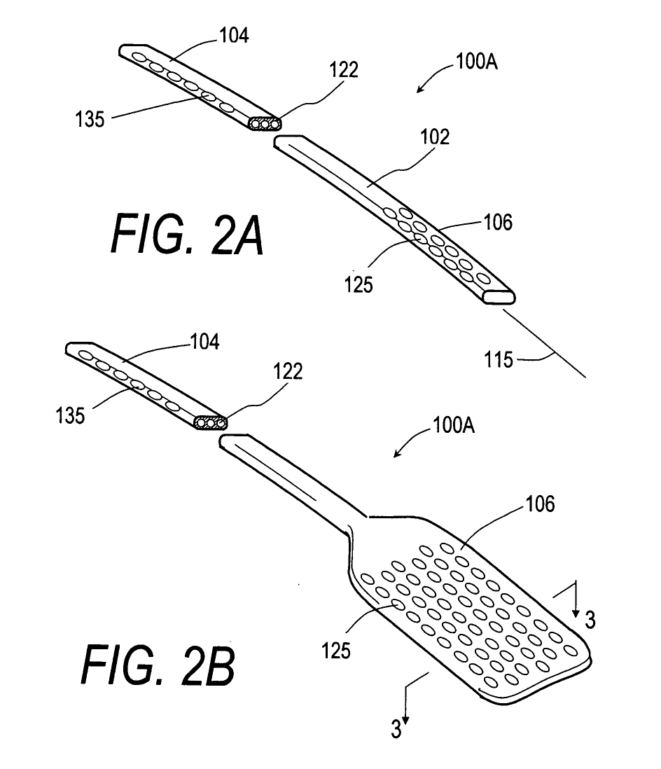

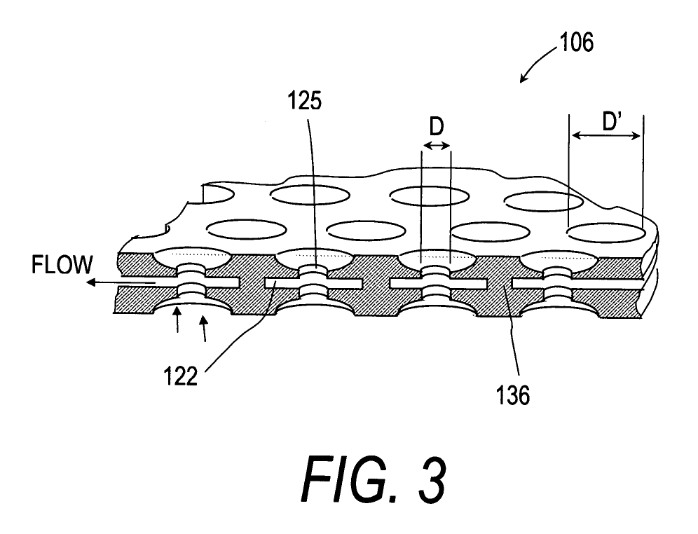

Lymphatic network stent for treating ocular hypertension. FIGS. 1, 2A and 2B illustrate a stent or implant 100A for carrying aqueous outflow to the lymphatic network of the eye, wherein the implant body 102 is fabricated at least partly of a shape memory polymer (SMP). The SMP body can be fabricated to have a first memory shape (FIG. 2B) that is compactable or deformable to a second temporary shape (FIG. 2A) to allow for a more minimally invasive entry path to implant the stent in the eye. The stent then expands to its memory shape in response to temperature or another selected stimulus.

In one preferred apparatus and method of the invention, referring to FIG. 1, the stent 100A has a first (outflow) end 104 that is sited within a region of the subconjunctival plane SCP generally radially inward of the lymphatic vessel network indicated at 105. The stent 100A has a second (inflow) end 106 that is sited within the interior of eye proximate the uveoscleral plane USP and ciliary body CB. The method of the invention includes the minimally invasive injection or implantation of stent 100A with a needle-like injector 110 shown in phantom view in FIG. 1.

The subconjunctival lymphatic network 105 has been described as comprising three regions or components. First, there are localized “nets” or networks of small lymphatic vessels indicated at LN in FIG. 1. Second, there are lymphatic circumferentials LC (FIG. 1) that extend 180° or more generally parallel to the limbus that drain the localized lymphatic nets LN. Third, the are two large lymphatic vessels LV (FIG. 1) at about 6 and 12 o'clock that drain the circumferential vessels LC and extend posteriorly within the scleral surface to drain the network and extend deep into the orbit. Daljit Singh, M.D. described the lymphatic system of the eye (see, as of Sep. 27, 2003: webpage http://www.ophmanagement.com/pf_article.asp?article=85458). It is believed that the subconjunctival lymphatic network can drain fluids rapidly when the outflow end 104 of the implant is optimally sited within the subconjunctival plane inward of the network LN. One objective and advantage of the invention is localization of the outflow end 104 of implant in an optimal site relative to the lymphatic network so that the eyes drainage network functions as a flow-limiting system to prevent excess aqueous flows that could result in hypotony. Hypotony is generally considered to be less than 6-10 mm Hg, and such low IOP can cause several distortions of the retina, lens and cornea that can degrade vision.

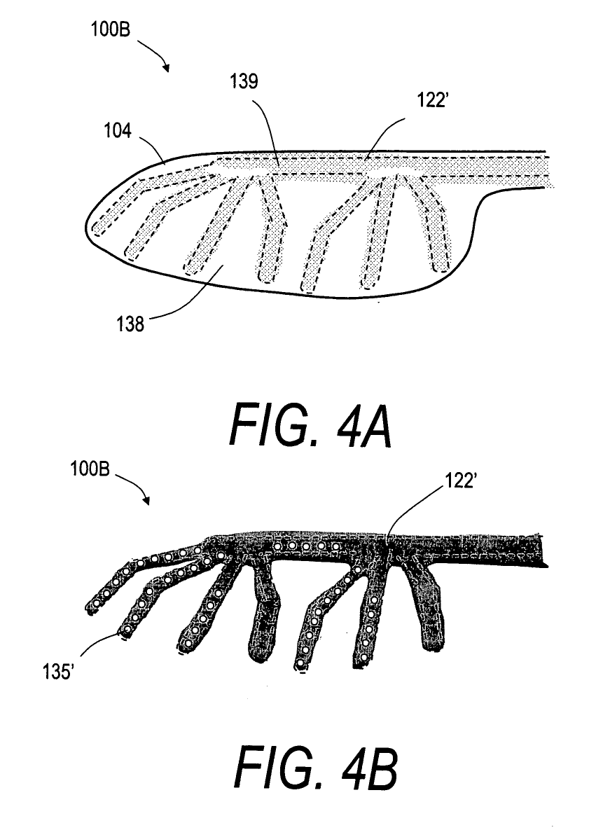

Now referring to FIGS. 2A and 2B, the body 102 of stent 100A extends along axis 115 and in one preferred embodiment is fabricated of a shape memory polymer (SMP) that is capable of the first compact cross-sectional shape of FIG. 2A and the second expanded cross-sectional shape of FIG. 2B. The scope of the invention encompasses any SMP implant body that has expandable first and/or second ends with flow pathways or channel(s) 122 extending therethrough. In FIG. 2A, the inflow end 106 is planar but also can be more bulb-shaped with at least one inflow aperture 125 therein. Preferably, a plurality of micron-scale apertures 125 are provided in the expanded cross-section inflow end 106 to provide for distributed inflow ports and to optionally serve as a filtering mechanism with micro- or nano-scale ports 125 (see FIG. 3). The inflow end 106 is adapted to expand at about body temperature and generate sufficient expansion forces to increase in surface area exposed in the plane, or to bluntly dissect somewhat about the uveoscleral plane USP. The apertures 125 range in dimension D across a principal axis from about 0.1 micron to about 50 microns. As can be seen in FIG. 3, the ports 125 can have a recessed or concavity with a larger dimension D′ to insure that tissue does not press directly against the port 125. More preferably, the apertures 125 range in dimension from about 0.5 micron to about 25 microns. The flow channel(s) 122 that extend between the first and second ends of stent 100A can range in cross-sectional dimension across a principal axis from about 0.1 micron to about 50 microns. More preferably, flow channel(s) 122 range in cross-sectional dimension between about 0.5 micron and 25 microns. In the exemplary embodiment of FIGS. 2A-2B, the apertures 135 in the outflow end 104 have dimensions similar to apertures 125. The outflow apertures 135 are shown as being oriented in one direction toward the lymphatic network, but also can be oriented about the entire surface of the implant. In another embodiment (not shown) the outflow end 104 also can have an expanded planar or bulb-like form of the shape memory polymer of the invention to expand the surface area of the outflow end and it ports. The implant preferably is fabricated of a transparent shape memory polymer.

The implant of FIGS. 2A and 2B can have any suitable length ranging from about 3 mm to 15 mm. The cross-section of the body 102 can be round, oval or rectangular and have a dimension across a principal axis from about 10 microns to 500 microns. Preferred dimensions in the range of less that about 200 microns will allow for simple injection of the implant into its preferred location. It should be appreciated that the shape memory polymer is used to increase the surface area—and inflow and/or outflow regions—of the stent and are optional while maintaining a reduced cross-section for introduction. Alternatively, the implant can be made of a non-shape memory polymer, a shape memory alloy (NiTi) or another biocompatible metal.

The stent of FIGS. 2A and 2B alternatively can have an expansion structure at its first or second end, 104 or 106, (or both ends) of a shape memory polymer in the form of a compacted open cell SMP foam material. Either type, as depicted in FIG. 2A, can be provided with a temporary reduced cross-sectional dimension. For example, the implant can be compacted to the form of a rod or similar shape for deployment from a needle, or an ultrathin constraining sleeve (e.g., also perforated) of a biodegradable material can be used. Thus, thermal or biodegradable means can be used to releasably maintain the stent 100A in the reduced cross-sectional shape.

In order to better describe stent 100A that is fabricated of SMPs, it is first useful to provide background on such shape memory polymers. SMPs demonstrate the phenomena of shape memory based on fabricating a segregated linear block co-polymer, typically of a hard segment and a soft segment. The shape memory polymer generally is characterized as defining phases that result from glass transition temperatures (T_{g}) in the hard and soft segments. The hard segment of SMP typically is crystalline with a defined melting point, and the soft segment is typically amorphous, with another defined transition temperature. In some embodiments, these characteristics may be reversed together with the segment's glass transition temperatures. Some SMPs that are suitable for the implant are a subset of shape memory polymer material that comprises a foam polymer. In one known use of such a foam SMP, the material has been more particularly identified as a CHEM (cold-hibernated elastic memory) polymeric foam that can be compacted.

Referring to FIG. 2B, the SMP form can be fabricated to provide a memory shape, such as 3-D shape of body portions 102 in FIG. 2B. In such an embodiment, when the SMP material is elevated in temperature above the melting point or glass transition temperature of the hard segment, the material is then formed into its memory shape. The selected shape is memorized by cooling the SMP below the melting point or glass transition temperature of the hard segment. When the shaped SMP is cooled below the melting point or glass transition temperature of the soft segment while the shape is deformed, that temporary shape is fixed. The temporary shape can be a highly compacted shape for introduction (such as a cylinder or helical form with shallow threads for axially or helically inserting into tissue) and maintained in the compacted state without a constraining sleeve member.

The original memory shape is recovered by heating the material above the melting point or glass transition temperature T_{g} of the soft segment but below the melting point or glass transition temperature of the hard segment. (Other methods for setting temporary and memory shapes are known which are described in the literature below). The recovery of the original memory shape is thus induced by an increase in temperature, and is termed the thermal shape memory effect of the polymer. The transition temperature can be body temperature or somewhat below 37° C. for a typical embodiment. Alternatively, a higher transition temperature can be selected and remote source can be used to elevate the temperature and expand the SMP structure to its memory shape (i.e., inductive heating or light energy absorption).

Besides utilizing the thermal shape memory effect of the polymer, the memorized physical properties of the SMP can be controlled by its change in temperature or stress, particularly in ranges of the melting point or glass transition temperature of the soft segment of the polymer, e.g., the elastic modulus, hardness, flexibility, permeability and index of refraction. Examples of polymers that have been utilized in hard and soft segments of SMPs include polyurethanes, polynorborenes, styrene-butadiene co-polymers, cross-linked polyethylenes, cross-linked polycyclooctenes, polyethers, polyacrylates, polyamides, polysiloxanes, polyether amides, polyether esters, and urethane-butadiene co-polymers and others identified in the following patents and publications: U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al. (all of which are incorporated herein by reference); Mather, Strain Recovery in POSS Hybrid Thermoplastics, Polymer 2000, 41(1), 528; Mather et al., Shape Memory and Nanostructure in Poly(Norbonyl-POSS) Copolymers, Polym. Int. 49, 453-57 (2000); Lui et al., Thermomechanical Characterization of a Tailored Series of Shape Memory Polymers, J. App. Med. Plastics, Fall 2002; Gorden, Applications of shape Memory Polyurethanes, Proceedings of the First International Conference on Shape Memory and Superelastic Technologies, SMST International Committee, pp. 120-19 (1994); Kim, et al., Polyurethanes having shape memory effect, Polymer 37(26):5781-93 (1996); Li et al., Crystallinity and morphology of segmented polyurethanes with different soft-segment length, J. Applied Polymer 62:631-38 (1996); Takahashi et al., Structure and properties of shape-memory polyurethane block copolymers, J. Applied Polymer Science 60:1061-69 (1996); Tobushi H., et al., Thermomechanical properties of shape memory polymers of polyurethane series and their applications, J. Physique IV (Colloque Cl) 6:377-84 (1996)) (all of the cited literature incorporated herein by this reference). The above background materials, in general, describe SMP in a non-open cell solid form. The similar set of polymers can be foamed, or can be microfabricated with an open cell structure for use in the invention. See Watt A. M., et al., Thermomechanical Properties of a Shape Memory Polymer Foam, available from Jet Propulsion Laboratories, 4800 Oak Grove Drive, Pasadena Calif. 91109 (incorporated herein by reference).

Shape memory polymers foams that fall within the scope of the invention typically are polyurethane-based thermoplastics that can be engineered with a wide range of glass transition temperatures. These SMP foams possess several potential advantages for intraocular implants, for example: very large shape recovery strains are achievable, e.g., a substantially large reversible reduction of the Young's Modulus in the material's rubbery state; the material's ability to undergo reversible inelastic strains of greater than 10%, and preferably greater that 20% (and up to about 200%-500%); shape recovery can be designed at a selected temperature between about 30° C. and 60° C. which will be be useful for the treatment system, and injection molding is possible thus allowing complex shapes. These polymers also demonstrate unique properties in terms of capacity to alter the material's water or fluid permeability and thermal expansivity. However, the material's reversible inelastic strain capabilities leads to its most important property-the shape memory effect. If the polymer is strained into a new shape at a high temperature (above the glass transition temperature T_{g}) and then cooled it becomes fixed into the new temporary shape. The initial memory shape can be recovered by reheating the foam above its T_{g}.

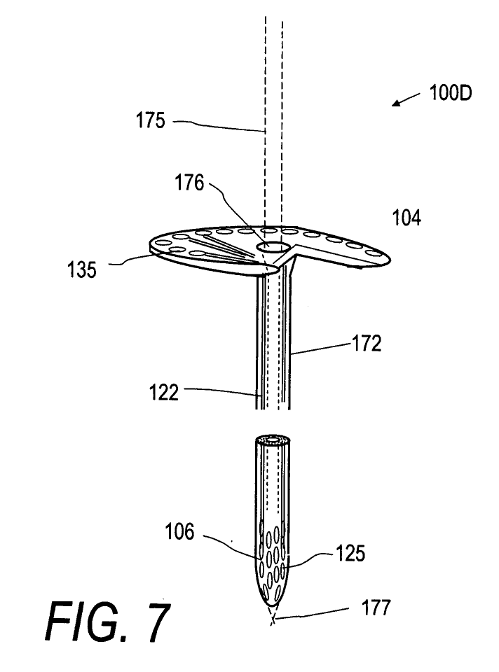

In any embodiment of polymer stent as in FIG. 1, 2 and 2B, the polymeric body 102 can be micro- or nano-fabricated using soft lithography techniques to provide an open or channeled interior structure to allow fluid flow therethrough. The shape of the apertures 125 and 135 and channels 122 (FIG. 3) can be molded in layers assembled by soft lithographic techniques. Such micro-apertures can be micro-fabricated of a resilient polymer (e.g., silicone) by several different techniques collectively known as soft lithography. For example, microtransfer molding is used wherein a transparent, elastomeric polydimethylsiloxane (PDMS) stamp has patterned relief on its surface to generate features in the polymer. The PDMS stamp is filled with a prepolymer or ceramic precursor and placed on a substrate. The material is cured and the stamp is removed. The technique generates features as small as 250 nm and is able to generate multilayer systems that can be used to fabricate the stent as well as lumen 120. Replica molding is a similar process wherein a PDMS stamp is cast against a conventionally patterned master. A polyurethane or other polymer is then molded against the secondary PDMS master. In this way, multiple copies can be made without damaging the original master. The technique can replicate features as small as 30 nm. Another process is known as micromolding in capillaries (MIMIC) wherein continuous channels are formed when a PDMS stamp is brought into conformal contact with a solid substrate. Then, capillary action fills the channels with a polymer precursor. The polymer is cured and the stamp is removed. MIMIC can generate features down to 1 μm in size. Solvent-assisted microcontact molding (SAMIM) is also known wherein a small amount of solvent is spread on a patterned PDMS stamp and the stamp is placed on a polymer, such as photoresist. The solvent swells the polymer and causes it to expand to fill the surface relief of the stamp. Features as small as 60 nm have been produced. A polymeric microstructure as in a stent can be entirely of a “Lincoln-log” type of assembly similar to that shown in Xia and Whitesides, Annu. Rev. Mater. Sci. 1998 28:153-84 at p. 170 FIG. 7d (the Xia and Whitesides article incorporated herein by reference).

FIG. 3 illustrates end portion 106 of a polymer stent 100A made by soft lithography techniques with the interior channel structure 122 maintained in an open form by posts 136. In one embodiment, the cross-sectional configuration of each aperture extends from surface dimension D′ (as described in micron ranges above) to a tapered down dimension as the aperture or port 125 transitions to the flow channel 122. The surface dimension D is selected so that tissue cannot collapse into the aperture to block fluid flows therein. The concavity of the aperture 125 provides an open space that will receive fluid flows that migrate through the tissue to the lower pressure within the aperture and interior channel 122. In any polymer embodiment of a stent, the polymer can have a “surface modification” to enhance fluid flows therethrough, and to prevent adherence of body materials to the surfaces of the outflow pathway.

While FIG. 1 shows the stent 100A implanted in a more-or-less radial or circumferential position, it should be appreciated that the scope of the invention includes any stent orientation wherein the stent's outflow end is positioned within, radially inward of, or generally proximate to the plane of the lymphatic network 105. The implant can be short or long and extend radially or circumferentially relative to the eye's optical axis from the uveoscleral plane to the subconjuctival site outward of the limbus.

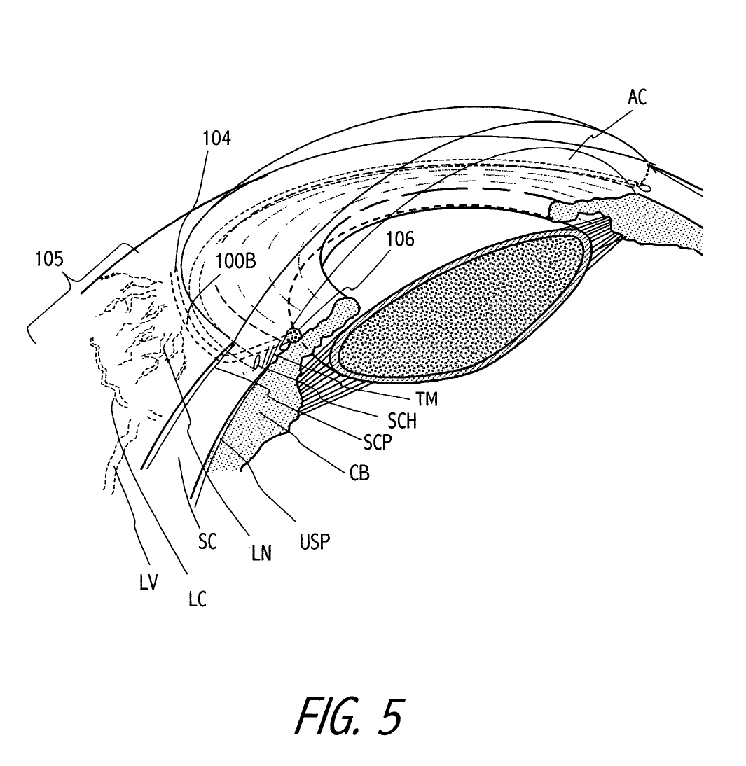

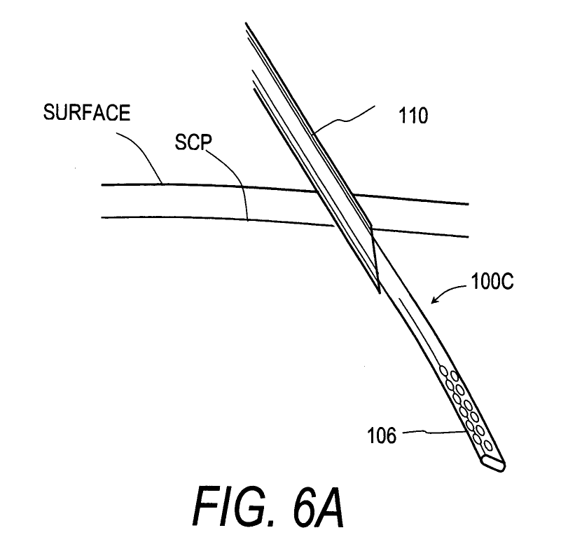

FIGS. 4A and 4B illustrate an alternative stent 100B that has a different embodiment of shape memory polymer outflow end 104. The SMP feature also can be used for the inflow end 106 of the stent. In this embodiment, at least a portion of the stent end portion is of a bioerodible shape memory polymer indicated at 138 in FIG. 4A. The network of outflow channels 122′ (and outflow ports 135′) are within a body portion of any polymer 139 or SMP that is non-biodegradable. As can be seen in FIG. 4B, the bioerodible SMP 138 is adapted to degrade over a selected time interval of days, weeks or months, to leave a branched polymer body carrying a network of outflow channels 122′. By this means, as seen in FIG. 4B, the outflow ports 135′ are distributed over a larger region of the lymphatic net without having a monolithic implant body. Thus, the bioerodible SMP 138, which can be a shape memory polymer foam, serves the function of expanding the branched body 139 that carries the network of outflow channels 122′ to a selected plan shape or configuration, wherein the bioerodible SMP 138 thereafter disappears. The same system can be used to expand the network of channels in the inflow end portion of the stent 100B. The expanded body carrying the network of lumens (FIG. 4B) can be designed to have any form branched system such as a fractal-based branched network. It should be further appreciated that the polymer body 139 also can be biodegradable, but adapted for degradation after a greatly extended life-time of the implant.

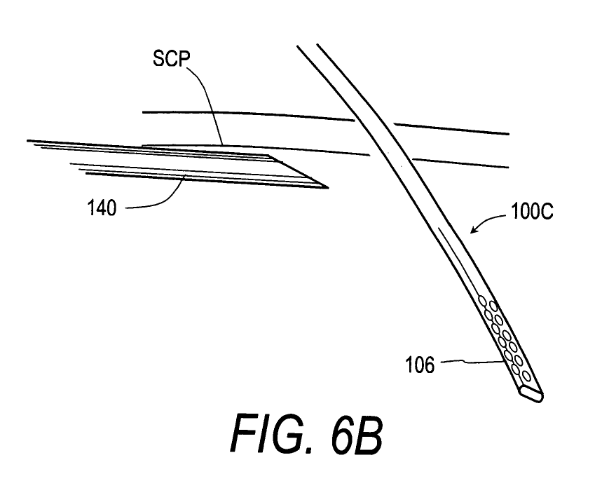

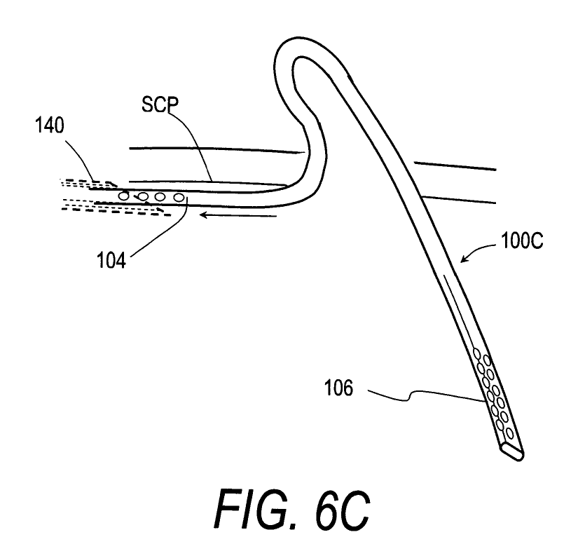

FIG. 5 shows another stent 100C corresponding to the invention wherein the inflow end 106 of the stent body is disposed within the anterior chamber AC, or within the trabecualr meshwork TM or interior of the cornea proximate the meshwork. In this embodiment, the bulb or planar end 106 has micron-scale apertures to serve as a filter. The implant of FIG. 5 can be implanted with a needle-like injector in one or two steps (radially and then circumferentially as in FIGS. 6A-6C) to provide the outflow end 104 of the stent in the desired location inward of the lympahtic net LN. The stent of FIG. 5 thus provides an elongated portion with substantial surface area and outflow apertures in the subconjuctival plane and within the targeted region of the lympthic network.

FIGS. 6A-6C illustrate the implantation of a flexible polymer stent with a first implantation path being at any angle through the sclera to reach the cilairy body (cf. FIG. 1) or anterior chamber AC (see. FIG. 5). Again, the objective of the method is to implant an elongated portion of the body with a subtantial surface area (having distributed, spaced apart outflow apertures) in the subconjuctival SCP plane proximate to the lympthic network. FIG. 6B showns a second implant path being created by sharp (or partly blunt dissection with member 140) in the subconjuctival plane. FIG. 6C depicts the flexible stent being “fished” back into the dissected path. In one embodiment, the outflow end 104 of the device is of a shape memory polymer that shortens axially after implantation in response to hydration or another stimulus (other than body temperature) to axailly retract into the dissected path. By this means, the elongated outflow end of the stent can be snipped off after being fished through the dissected subconujctival path—and its will controllably retract into the incision a selected distance.

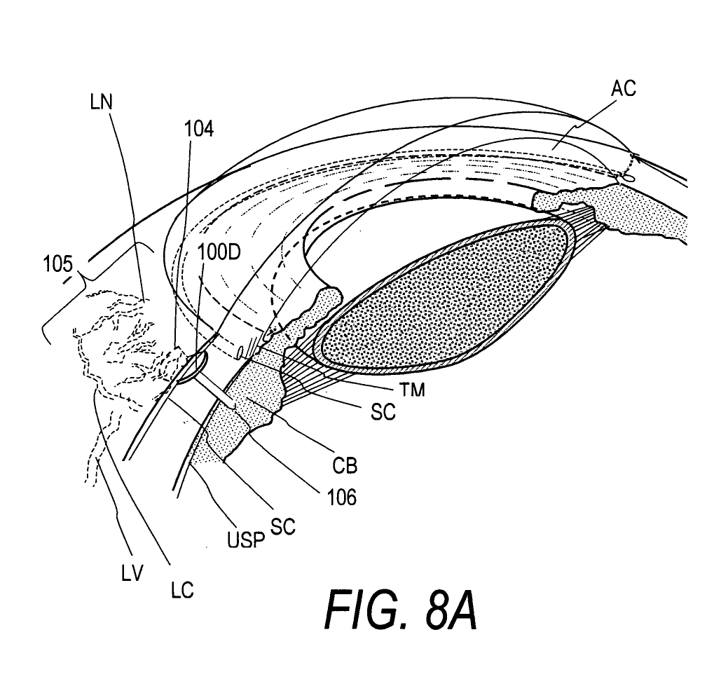

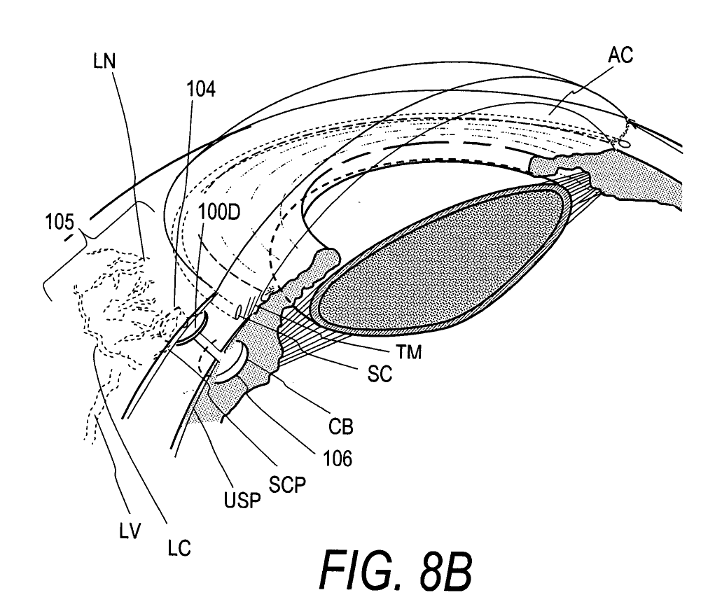

FIG. 7 depicts an alternative stent 100D that has a part disc-shaped outflow end 104 with a notch 170 that allows the end to be rotated into a dissected subconjunctival plane through a minimally dimensioned needle-size penetration (see FIG. 8A). The stent 100D that can be introduced in an axial direct penetration and the length of the medial portion 172 (at any selected angle) will control the depth to localize the inflow end 106 in the uveoscleral plane or somewhat withn the ciliary body CB. A needle introducer 175 can extend through bore 176 in the stent for introduction with the sharp tip 177 extending therethorugh. The flow channels 122 in the stent can be microfabricated as described above to communicate between the pores 125 and 135 in the ends of the stent. A plurality of such stents 100D can be implanted. Alternatively, the stent body can be an open cell polymer that is surface modified to provide a non-stick surface for maintainign the flow means. Alternatively, the bore 176 in the stent body can serve as a flow channel. The medial portion 172 of the stent also can be carried in a slot in a needle like introducer member and fall within the scope of the invention. FIG. 8B illustrates the stent 100D after implantation with the inflow end 106 of a shape memory polymer having expanded to a flattened memory shape.

It should be appreciated that an alternative stent (not shown) can be similar to FIG. 7 but have a helical medial portion 172 for helical introduction and for providing a greater microperforated inflow surface in the region of the uveoscleral plane USP. The stent of the invention can further be coupled with implant bodies for retracting the region of the trabecular meshwork as disclosed in the following applications: Ser. No. 60/425,969 filed Nov. 13, 2002 (Docket No. S-AEG-002) titled Implants and Methods for Treating Glaucoma; and Ser. No. 60/424,869 filed Nov. 7, 2002 (Docket No. S-AEG-001) titled Implants and Methods for Treating Glaucoma, the specifications of which are incorporated herein in their entirely by this reference.

Any of the stents of the invention also can have microfabricated one-way valves in the microchannels of the stent. The stents of the invention also can carry a surface coating of a bioerodible polymer that releases a selected drug, for example of the type that enhances uveoscleral aqueous flows, or that limits aqueous production (e.g., latanoprost, timolol).

In order to locate the lymphatic network, the method of the invention can included the injection of a dye which can be seen filtering through the vessels of the lymphatic net.

In another embodiment, and stent body can have a plurality of flow channels within a monolith of a photomodifiable polymer or a shape memory polymer. The photomodifiable body portion can carry a chromophore for targeting with a selected wavelength of light to thermally heat the polymer portion (above body temperature) to release its stored energy to move it toward a memory shape, for either increasing or decreasing the cross-section of the flow pathway. Alternatively, different sections of the flow pathway(s) can be targetable to increase or decrease the flows therethrough. The stent body can further carry one or more markers that respond to light energy (e.g., by reflectance) to allow localization of energy on the targeted photomodifiable polymer for post-implant outflow control. In a related embodiment, the implant body can have entirely collapsed flow channels that remain in reserve until a surrounding photomodifiable polymer or SMP is photo-actuated to cause an increase in the cross-section of the flow channel. By this means, a new channel can opened if other channels become degraded by body accumulations in the first-used channels.

The scope of the invention includes any type of polymer surface modifications that are known in the art, to prevent accumulations within the stent's flow channels, including new technologies on the horizon. For example, researchers have used a unique compound called mussel adhesive protein which contains a high concentration of an amino acid, DOPA (dihydroxyphenylalanine) in combination with a DOPA molecule (a well-known repellant molecule) of a polyethylene glycol (PEG). The researchers demonstrated that the new compound could be easily attached to common implant surface materials to render the surfaces resistant to cell attachment for a period of time (see: http://www.eurekalert.org/pub_releases/2003-04/acs-cdf040703.php).

The scope of the invention includes any electroless plating of the polymer lumen surfaces as are known in the art to prevent cell adherence and clogging of the stent's flow surfaces, including the ultrathin biocompatible coatings of any metal, such as platinum, gold, tantalum and the like.

Those skilled in the art will appreciate that the exemplary embodiments and descriptions thereof are merely illustrative of the invention as a whole. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention.

What is claimed is:

1. A method for treating ocular hypertension in a human eye comprising implanting a stent having an interior flow passageway between a region of the subconjuctival plane proximate the lymphatic vessel network and a region of the uveoscleral plane.

2. A method as in claim 1 wherein the stent is implanted to extend from a portion of the subconjunctival plane radially inward of the circumferential lymphatic vessels to the region of the uveoscleral plane.

3. A method as in claim 2 wherein the stent is implanted to extend within the subconjunctival plane proximate the limbus at about 6 o'clock or 12 o'clock.

4. A method as in claim 1 wherein the stent is provided with a shape memory polymer end portion that is capable of moving to its memory shape in the uveoscleral plane.

6. A method as in claim 1 wherein the stent is provide with a shape memory polymer end portion that is capable of moving to its memory shape in the subconjunctival plane.

7. A method for controlling intraocular pressure (IOP), the method comprising implanting a stent body with at least one flow channel therein between the interior of the eye and the subconjunctival plane proximate the lymphatic vessel network wherein the lymphatic system naturally controls outflows and IOP.

8. A method for controlling IOP as in claim 7 wherein the implanting step provides a fenestrated outflow end in the stent body that extends generally circumferentially in the subconjunctival plane proximate the lymphatic network.

9. A method for controlling IOP as in claim 7 including the step of identifying the lymphatic network by dye injection prior to the implanting step for selecting the site for the outflow end of the stent body.

10. A method for controlling IOP as in claim 7 further including the step of implanting a fenestrated inflow end of the stent in a site selected from the uveoscleral plane, anterior chamber, ciliary body, the region of Schlemm's canal, or within tissue about the angle of the anterior chamber.

11. A stent for treating ocular hypertension in a human eye comprising a stent body of a polymer, the stent body having a first end and a second end with at least one flow pathway extending between openings in said first and second ends, the stent body dimensionally configured for extending from a region of the subconjunctival plane proximate to the lymphatic vessel network to the interior of the eye.

12. A stent as in claim 11 wherein the stent body is at least partially of a shape memory polymer.

13. A stent as in claim 11 wherein the stent body has at least one end portion of a shape memory polymer capable of a temporary reduced cross-sectional shape and a memory expanded cross-sectional shape.

14. A stent as in claim 11 wherein the stent body has at least one end portion with a plurality of micro-apertures that communicate with the at least one flow pathway.

15. A stent as in claim 11 wherein the micro-apertures have a dimension across a principal axis ranging from 0.1 to 25 microns.

16. A stent as in claim 11 wherein the stent body is of a transparent polymer.

17. A stent as in claim 13 wherein a section of a least one end portion of the stent body is of a biodegradable shape memory polymer.

18. A lumened stent for reducing ocular hypertension, the stent body of a shape memory polymer (SMP) dimensionally configured for extending between the subconjunctival plane inward of the eye's lymphatic drainage system and a region of the uveoscleral plane.

19. A stent as in claim 18 wherein at least one flow pathway within the stent body is within a photo-modifiable polymer portion for post-implant modification of the flow capacity of the at least one pathway.

20. A stent as in claim 18 wherein the stent body carries at least one light-responsive marker element for localizing the photo-modifiable polymer portion.

21. A method of making a stent for treating ocular hypertension, the method comprising utilizing soft lithographic polymer microfabrication means to assemble a stent with micro-fenestrated first and second stent ends with at least one interior flow channel communicating with the micro-fenestrations of the first and second ends.

22. A method of fabricating a biomedical stent as in claim 21 wherein the stent is microfabricated at least partly of a shape memory polymer.
